(19) United States
(12) Patent Application Publication
(10) Pub. No.: US 2017/0097400 A1
(43) Pub. Date: Apr. 6, 2017

(54) MAGNETIC RESONANCE IMAGING APPARATUS AND METHOD FOR GENERATING WATER-FAT SEPARATION IMAGE

(71) Applicant: HITACHI, LTD., Tokyo (JP)

(72) Inventor: Norimasa NAKAI, Tokyo (JP)

(21) Appl. No.: 15/311,630

(22) PCT Filed: Jun. 10, 2015
(86) PCT No.: PCT/JP2015/066694

§ 371 Date: Nov. 16, 2016

(30) Foreign Application Priority Data
Jun. 12, 2014 (JP) 2014-121119

(51) Int. Cl. G01R 33/561; A61B 5/00; G01R 33/48; A61B 5/055

(52) U.S. Cl. CPC G01R 33/561; A61B 5/055; A61B 5/742; A61B 5/7257; G01R 33/4828; A61B 2576/00

(57) ABSTRACT

In order to provide a magnetic resonance imaging apparatus that can acquire an image capable of quantitative assessment of fat and a method for generating water-fat separation images, echo signals are acquired during application of a frequency encoding gradient magnetic field of positive and negative polarities at the same echo time, a correction amount for correcting an adverse effect of reception frequency characteristics is evaluated from the pair of correcting echo signals, and then the correction amount is used for removing the adverse effect of reception frequency characteristics of positive- and negative-polarity images acquired by inverting a polarity of the frequency encoding gradient magnetic field.

### TECHNICAL FIELD

The present invention relates to a magnetic resonance imaging (hereinafter, referred to “MRI”) apparatus that measures a nuclear magnetic resonance (hereinafter, referred to “NMR”) signal of a hydrogen atomic nucleus (hereinafter, referred to “proton”) included in an object, and, in particular, to a technique for acquiring an image for quantitative assessment of fat.

### BACKGROUND ART

An MRI apparatus is an apparatus for measuring NMR signals that atomic nuclear spin comprising tissues of an object, particularly a human body, generates due to external magnetic field variation in order to two- or three-dimensionally visualize shapes of the head, abdomen, limbs, and the like. In imaging, the NMR signals are provided with phase encoding that is different according to a gradient magnetic field, and then frequency encoding is performed on the NMR signals to be measured as time-series data. A two- or three-dimensional Fourier transform is performed on the measured NMR signals in order to reconstruct an image.

An MRI apparatus can acquire images having various tissue contrasts by changing parameters such as an echo time (hereinafter, referred to “TE”) and a repetition time (hereinafter, referred to “TR”) and performing image calculation. Clinically, an image in which signals from fat tissues are restricted is requested in many cases. A method for obtaining a plurality of images whose TEs are different in order to acquire an image in which water and fat are separated by calculation is taken as an example of a method for acquiring the image in which signals from fat tissues are restricted. As the typical method, used is a method described in Non-Patent Literature 1 (hereinafter, referred to “DIXON technique”).

In an MRI apparatus, caused are spatial inhomogeneity in a static magnetic field caused by a magnet structure and spatial inhomogeneity in a static magnetic field caused by that magnetic sensitivity varies for each site of an object placed in a static magnetic field space (hereinafter, collectively referred to as “static magnetic field inhomogeneity”). Non-Patent Literature 2 discloses a two-point DIXON technique with static magnetic field correction in which a function of correcting an effect of the static magnetic field inhomogeneity was added to the DIXON technique.

### CITATION LIST

### Non-Patent Literature

NPTL 1: W. Thomas Dixon “Simple Proton Spectroscopic Imaging” RADIOLOGY, Vol. 153, p. 189-194, (1984)

NPTL 2: Bernard D. Coombs “Two-Point Dixon Technique for Water-Fat Signal Decomposition with BO Inhomogeneity Correction” Magnetic Resonance in Medicine, vol. 38, p.884-889, (1997)

### SUMMARY OF INVENTION

### Technical Problem

In order to quantify fat, for example, high-speed imaging is required during respiratory arrest when the hepatic region is imaged. At this time, after receiving a first TE signal, the next TE signal needs to be received continuously by inverting a polarity of a frequency encoding gradient magnetic field.

In an image acquired from signals of the first TE and the next TE, an adverse effect of reception frequency characteristics can be generated inversely in the positive polarity and negative polarity.

The reception frequency characteristics are a characteristic of a receiver whose sensitivity (gain) differs according to a frequency to be received, and an echo signal strength varies according to the frequency to be received even when an echo signal to be generated has the same signal strength. The reception frequency characteristics differ depending on connected reception coils and a target to be imaged.

When the adverse effect of reception frequency characteristics is generated inversely in the positive polarity and negative polarity, the adverse effect of reception frequency characteristics is found in a frequency encoding direction of an image. NPTL 2 does not disclose a function of correcting the adverse effect of reception frequency characteristics. Therefore, it is difficult to generate a water-fat separation image at a level capable of performing quantitative assessment on fat from the image acquired from the two TE signals using the DIXON technique of NPTL 2.

On the other hand, when the first TE signal and the next TE signal are measured in a frequency encoding gradient magnetic field of the same polarity in order to avoid the adverse effect of reception frequency characteristics, imaging time extension, reduction of the number of imaging slices, and the like are restricted.

A purpose of the present invention is to provide a magnetic resonance imaging apparatus that can acquire an image capable of quantitative assessment of fat by removing an adverse effect of reception frequency characteristics from an image acquired by inverting a frequency encoding gradient magnetic field and a method for generating water-fat separation images.

### Solution to Problem

In order to achieve the above purpose, the present invention acquires an echo signal during application of a frequency encoding gradient magnetic field of positive polarity and acquires an echo signal during application of a frequency encoding gradient magnetic field of negative polarity in the same TE. A correction amount for correcting an adverse effect of reception frequency characteristics is evaluated from these signals, and the correction amount is used for correcting a signal strength of an image derived from the TE signals acquired by inverting a polarity of the frequency encoding gradient magnetic field.

Specifically, the MRI apparatus of the present invention has characteristics shown as follows.

The MRI apparatus of the present invention is characterized by being provided with static magnetic field magnets; high frequency generation units that generate high-frequency magnetic field pulses; reception units that include high-frequency coils receiving echo signals to be generated by nuclear magnetic resonance; gradient magnetic field coils; a control unit that controls the high frequency generation units, the gradient magnetic field coils, and the reception units according to a predetermined pulse sequence; and a signal processing unit that processes the echo signals, by that the pulse sequence is a multi-echo sequence that acquires echo signals during application of frequency encoding gradient magnetic fields of different polarities at a plurality of echo times after being excited by the high-frequency magnetic field pulses, and by that the signal processing unit generates correction data using a pair of correcting echo signals acquired during the application of frequency encoding gradient magnetic fields of positive and negative polarities at the same echo time and is provided with a correction unit that corrects echo signals acquired during the application of frequency encoding gradient magnetic fields of different polarities.

The correction unit is characterized by generating the correction data using echo signals acquired in a correction data measuring sequence to be executed separately from the multi-echo sequence.

The correction data measuring sequence is characterized by being the same type as the multi-echo sequence other than an application condition of a phase encoding gradient magnetic field.

The pair of correcting echo signals is characterized by being echo signals acquired in the executed multi-echo sequence and echo signals acquired in a correction data measuring sequence to be executed separately from the multi-echo sequence.

The pair of correcting echo signals is characterized by being signals acquired by applying a low-frequency phase encoding gradient magnetic field.

The high-frequency coil is characterized by comprising a plurality of small coils, and the correction unit is characterized by generating correction data for each of the small coils in order to perform correction on echo signals received by each of the small coils using the correction data.

The correction unit is characterized by evaluating the correction data from a ratio of data acquired by performing a two-dimensional Fourier transform on the pair of correcting echo signals for each of the small coils.

The multi-echo sequence is characterized by being a water-fat separation sequence that acquires echo signals at a first echo time in which echo signals from water and echo signals from fat are in the same phase and at a second echo time in which the echo signals from water and the echo signals from fat are in the reverse phase in the frequency encoding gradient magnetic fields of different polarities.

The echo signals used for generating the correction data is characterized by being echo signals acquired at an echo time same as the first echo time or the second echo time.

The water-fat separation sequence is characterized by setting the first echo time longer than the second echo time.

Also, the water-fat separation image generating method of the present invention has the following characteristics.

The water-fat separation image generating method is used for generating a plurality of types of images using echo signals to be generated by nuclear magnetic resonance, the echo signals are acquired during application of frequency encoding gradient magnetic fields of different polarities at a plurality of echo times after being excited by a high-frequency magnetic field pulse, and then the echo signals acquired during the application of frequency encoding gradient magnetic fields of different polarities are corrected using a pair of correcting echo signals acquired during application of frequency encoding gradient magnetic fields of positive and negative polarities at the same echo time.

The pair of correcting echo signals is characterized by being signals acquired by applying a low-frequency phase encoding gradient magnetic field.

The echo signals are collected at the first echo time in which echo signals from water and echo signals from fat are in the same phase and at the second echo time in which the echo signals from water and the echo signals from fat are in the reverse phase in order to generate a plurality of types of images using first echo signals acquired at the first echo time and second echo signals acquired at the second echo time.

The pair of correcting echo signals is characterized by being signals acquired by applying a low-frequency phase encoding gradient magnetic field at an echo time same as the first echo time or the second echo time.

A fat distribution ratio is characterized by being calculated using the plurality of types of images.

### Advantageous Effects of invention

According to the present invention, an adverse effect of reception frequency characteristics can be removed in an image (hereinafter, referred to as “an image of the positive polarity”) acquired from echo signals (hereinafter, referred to as “echo signals of the positive polarity”) acquired during application of the frequency encoding gradient magnetic field of the positive polarity by inverting a frequency encoding gradient magnetic field and in an image (hereinafter, referred to as “an image of the negative polarity”) acquired from echo signals (hereinafter, referred to as “echo signals of the negative polarity”) acquired during application of the frequency encoding gradient magnetic field of the negative polarity by inverting a frequency encoding gradient magnetic field. By removing the adverse effect of reception frequency characteristics, for example, accuracy in quantitative assessment of fat can be improved even in a case of using images acquired by imaging the hepatic region or the like at a high speed during respiratory arrest. Also, in imaging to acquire images for the quantitative assessment of fat, the imaging time can be reduced, or the number of imaging slices can be increased.

### BRIEF DESCRIPTION OF DRAWINGS

FIG. 1 is a block diagram illustrating an overall configuration of an MRI apparatus to which the present invention is applied.

FIG. 2 is a block diagram illustrating a configuration of a signal processing unit of the MRI apparatus to which the present invention is applied.

FIG. 3 illustrates a gradient echo (GrE)-type sequence to be used in first and second embodiments.

FIG. 4 shows an example of reception frequency characteristics of a reception coil.

FIG. 5 illustrates an effect of the reception frequency characteristics on an image.

FIG. 6 shows an example of a correction data measuring sequence to be used in the first embodiment.

FIG. 7 illustrates a processing flow chart for correcting the reception frequency characteristics.

FIG. 8 illustrates a processing flow chart for combining channels to evaluate an image of fat contents.

FIG. 9 shows an example of phantom images acquired in a gradient echo (GrE)-type sequence.

FIG. 10 shows an example of phantom images that were acquired from each channel of the reception coils in the gradient echo (GrE)-type sequence.

FIG. 11 shows correction data on which a two-dimensional Fourier transform was performed.

FIG. 12 illustrates graphs showing a ratio of the correction data on which fitting was performed with the ratio of the correction data.

FIG. 13 shows an example of the correction data measuring sequence to be used in the second embodiment.

FIG. 14 illustrates the gradient echo (GrE)-type sequence to be used in a third embodiment.

### DESCRIPTION OF EMBODIMENTS

Hereinafter, the embodiments of the present invention will be described using the drawings. It is noted that the same reference numerals are provided for the same functions in all the drawings and the repeated descriptions are omitted.

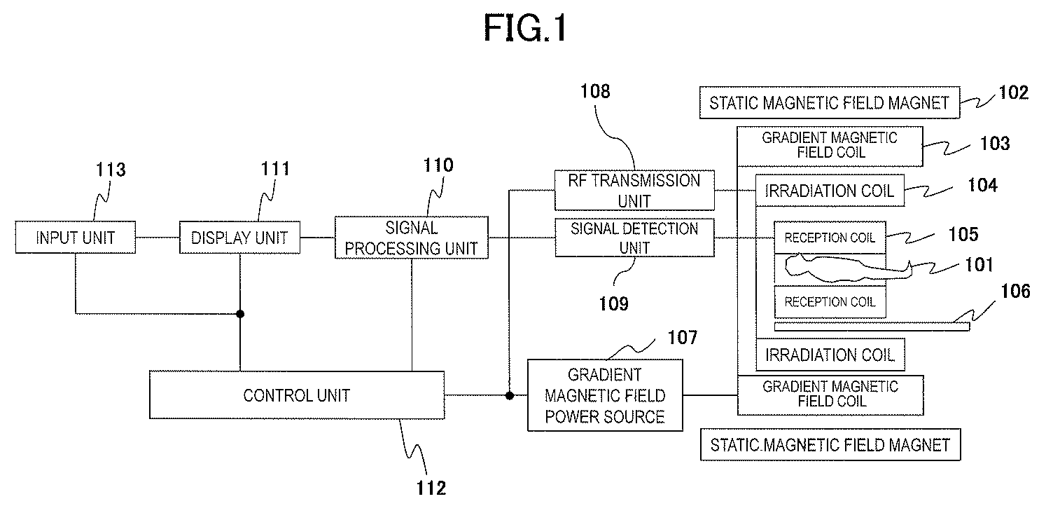

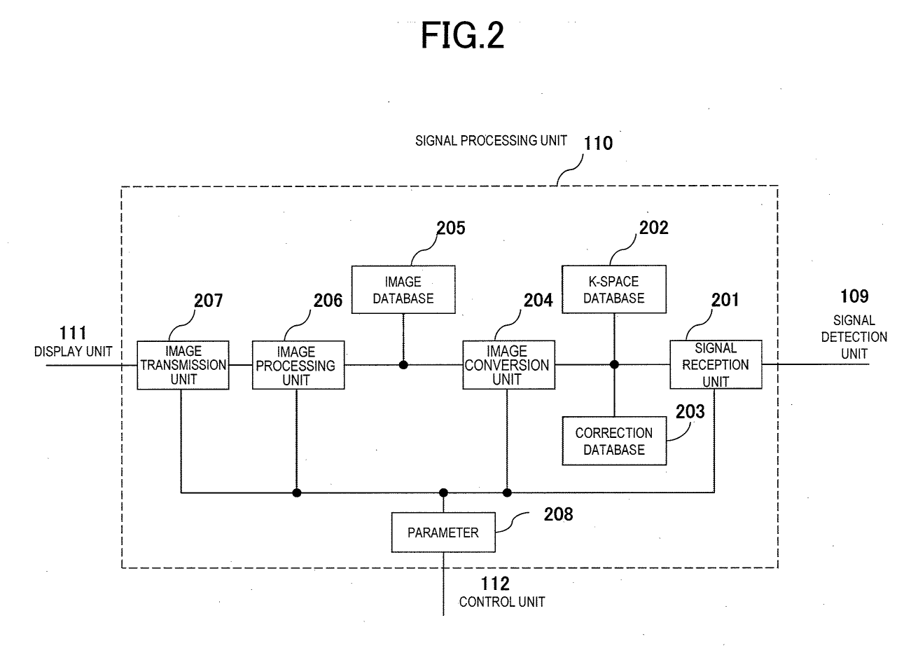

First, based on FIGS. 1 and 2, the overall configuration of the MRI apparatus to which the present invention is applied and the signal processing unit of the apparatus will be described respectively.

FIG. 1 is a block diagram illustrating the overall configuration of the MRI apparatus to which the present invention is applied. The MRI apparatus to which the present invention is applied comprises static magnetic field magnets 102 that generate a static magnetic field around an object 101; gradient magnetic field coils 103 that generate gradient magnetic fields; irradiating high-frequency coils (hereinafter, referred to as “irradiation coils”) 104 that irradiate high-frequency magnetic field pulses (hereinafter, referred to as “RF pulses”) to the object 101; receiving high-frequency coils (hereinafter, referred to as “reception coils”) 105 that receive NMR signals from the object; a bed 106 on which the object 101 lies; a gradient magnetic field power source 107 that transmits signals to the gradient magnetic field coils 103 for generating gradient magnetic fields; an RF transmission unit 108 that transmits signals for generating RF pulses to the irradiation coils 104; a signal detection unit 109 that detects echo signals received through the reception coils 105; a signal processing unit 110 that processes signals detected from the signal detection unit 109; a display unit 111 that displays images and the like; a control unit 112 that controls imaging and the like; and an input unit 113 that inputs parameters and the like required for imaging.

It is noted that the irradiation coils 104 and the RF transmission unit 108 are collectively referred to as a high frequency generation unit and the reception coils 105 and the signal detection unit 109 are collectively referred to as a reception unit.

The static magnetic field magnets 102 are formed of any of permanent magnets, superconducting magnets, and normal conducting magnets, are disposed in a sufficient space around the object 101, and generate a homogeneous static magnetic field in a direction parallel or vertical to the body axis of the object 101.

The gradient magnetic field coils 103 apply gradient magnetic fields of the three-axis directions X, Y, and Z to the object 101 according to the signals from the gradient magnetic field power source 107. According to the method of applying the gradient magnetic fields, an imaging cross section of the object is determined, and phase encoding and frequency encoding are provided for the signals.

The irradiation coils 104 generate RF pulses according to the signals of the RF transmission unit 108. The RF pulses excite protons included in biological tissues in an imaging cross section of the object 101 set by the gradient magnetic field coils 103 in order to induce an NMR phenomenon.

The reception coils 105 receive echo signals generated by the NMR phenomenon of the protons included in the object 101 induced by the RF pulses irradiated from the irradiation coils 104. Although the reception coils 105 may have one coil, the reception coils 105 may be formed of one multi-channel coil in which a plurality of small coils are combined (such as a multiple array coil and a phased array coil).

The signal detection unit 109 detects echo signals received through the reception coils 105 disposed closely to the object 101. In a case where a reception coil includes a plurality of coils (channels), the echo signals are detected for each channel.

The signal processing unit 110 performs signals processing on the echo signals detected by the signal detection unit 109 in order to generate an image of the object 101. The details of the signal processing unit 110 will be described as follows using FIG. 2.

The display unit 111 displays images generated by the signal processing unit 110 and imaging parameters.

The input unit 113 is used for an operator to input parameters such as TRs and TEs required for imaging. The input parameters are displayed on the display unit 111, are transmitted to the control unit 112, and are used for controlling imaging.

The control unit 112 generates a predetermined pulse sequence for repeatedly generating each of gradient magnetic fields and RF pulses that perform slice selection, phase encoding, and frequency encoding based on parameters input from the input unit 113 in order to control the gradient magnetic field power source 107, the RF transmission unit 108, and the signal processing unit 110.

The pulse sequence includes a main measurement pulse sequence for main measurement and a correction data measuring sequence for measuring correction data.

FIG. 2 illustrates a configuration of the signal processing unit 110 of the MRI apparatus of the present embodiments. The signal processing unit 110 comprises a signal reception unit 201, an image conversion unit 204, an image processing unit 206, and an image transmission unit 207. Also, the signal processing unit 110 is provided with a memory (a k-space database 202, a correction database 203, and an image database 205) that accommodates data acquired in each of these units and a memory (memory (parameters) 208) that accommodates data acquired from the control unit.

Each of these units can be comprised of a CPU and a memory. The memory previously stores a program for executing functions of these units, and the CPU reads and executes the program of the memory. As the result, operations of these units can be realized.

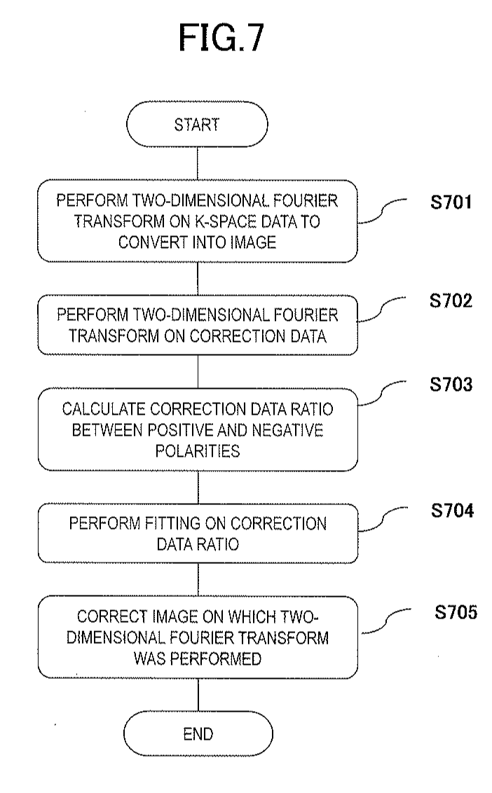

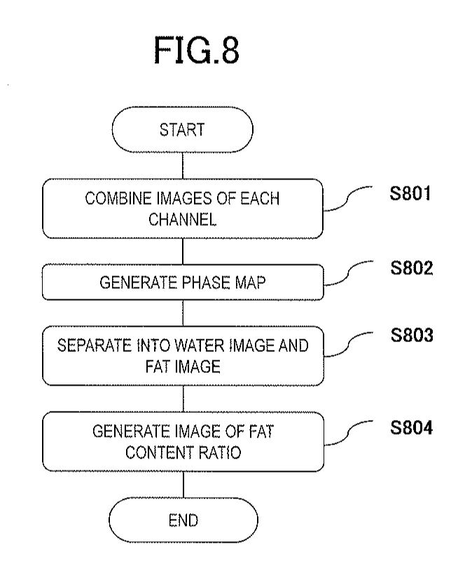

For example, the memory previously stores programs illustrated in the flows of FIGS. 7 and 8. The CPU reads and executes the program illustrated in the flow of FIG. 7 from the memory, which executes operations of the image conversion unit 204. Also, the CPU reads and executes the program illustrated in the flow of FIG. 8 from the memory, which executes operations of the image processing unit 206.

Hereinafter, although processing procedures of the image conversion unit 204 and the image processing unit 206 will be described as that to be realized as software, the present embodiment is not limited to the software, and processes of the image conversion unit 204 and the image processing unit 206 can also be realized by hardware such as ASIC and FPGA.

Of echo signals detected by the signal detection unit 109, the signal reception unit 201 stores signals acquired in main measurement in the k-space database 202 based on information of arrangement in k-space. On the other hand, of echo signals detected by the signal detection unit 109, the signal reception unit 201 stores a pair of correcting echo signals acquired in correction data measurement or signals acquired in low-frequency phase encoding of the main measurement and signals acquired in the correction data measurement in a case of using the signals acquired in the main measurement in the correction database 203 based on the information of arrangement in k-space.

The image conversion unit 204 performs a Fourier transform on the k-space data stored in the k-space database 202 in order to convert into an image, corrects reception frequency characteristics using the correction data stored in the correction database 203, and stores the image in the image database 205. This correction is made for each of coils. For example, in a case of reception using a reception coil having a plurality of small coils (channels), the correction is made for each of the small coils.

The image processing unit 206 performs image processing on the image stored in the image database 205.

The image processing includes, for example, combining images of each channel of the reception coils, generating water images and fat images, correcting sensitivity unevenness of the reception coils 105, and the like. The image processing unit 206 transmits the processed images to the image transmission unit 207.

The image transmission unit 207 transmits the images on which image processing was performed by the image processing unit 206 to the display unit 111. The images to be transmitted include In-phase images, Out-of-phase images, water images, fat images, images showing fat content ratios, and the like.

The parameters to be stored in the memory 208 include information of slice encoding, frequency encoding, and phase encoding in a pulse sequence that the signal reception unit 201 requires; image matrices that the image conversion unit 204, the image processing unit 206, and the image transmission unit 207 require; parameters for filtering and the like; and control information, and the memory 208 acquires these parameters from the control unit 112.

As described hereinbefore, the embodiments of the present invention described using FIGS. 1 and 2 are common to an embodiment to be described below.

Hereinafter, an operation procedure through which the present invention works and processes in the signal processing unit will be specifically described using a pulse sequence.

### First Embodiment

In the present embodiment, a main measurement pulse sequence and a correction data measuring sequence that measures correction data for correcting data acquired in the main measurement are executed under control by the control unit 112.

The main measurement pulse sequence is a multi-echo sequence for measuring echo signals each time a frequency encoding gradient magnetic field pulse is inverted in order to acquire a plurality of images with different TEs.

The correction data measuring sequence acquires the correction data for removing an adverse effect of reception frequency characteristics to be included in each of echoes in the main measurement pulse sequence and is a pulse sequence in which a polarity of a frequency encoding gradient magnetic field in the main measurement pulse sequence is inverted by applying a phase encoding gradient magnetic field at low frequencies only.

In the present embodiment, signals acquired in the main measurement and echo signals acquired in the correction data measurement are used as a pair of correcting echo signals that are used as correction data and acquired during application of frequency encoding gradient magnetic fields of different polarities in the same TE.

First, described will be the main measurement pulse sequence and the adverse effect of reception frequency characteristics included in the echo signals measured in the main measurement pulse sequence.

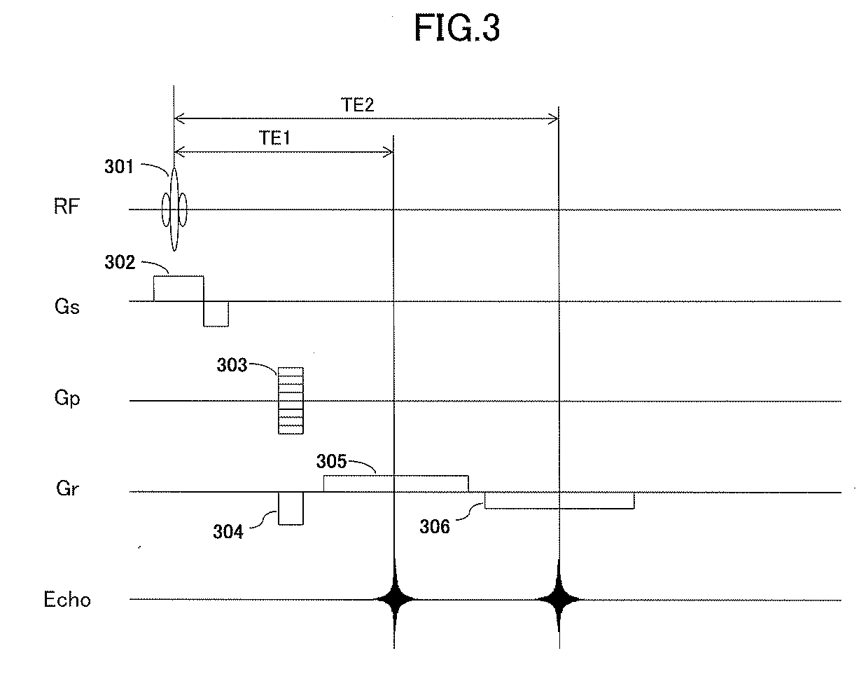

FIG. 3 shows an example of the main measurement pulse sequence. This pulse sequence acquires two types of image data with different TEs and a gradient echo (GrE)-type sequence method. This pulse sequence acquires the two types of image data with the different TEs by inverting a frequency encoding gradient magnetic field from the positive polarity to the negative polarity. Typically, this pulse sequence is applied to water-fat separation imaging.

The control unit 112 performs the following control and executes this pulse sequence. First, a slice selecting gradient magnetic field 302 is applied simultaneously with irradiation of an RF pulse 301 in order to excite only a desired tomographic plane. Next, a phase encoding gradient magnetic field 303 is applied for encoding positional information, and a negative-direction frequency encoding gradient magnetic field (pre-pulse) 304 is applied simultaneously. Then, a positive-direction frequency encoding gradient magnetic field 305 is applied in order to generate a first echo signal after TE1 from the RF pulse. Next, a negative-direction frequency encoding gradient magnetic field 306 is applied again in order to generate the next echo signal after TE2 from the RF pulse.

Such a sequence is repeatedly executed by the number of times of phase encoding while changing an application amount of the phase encoding gradient magnetic field 303 in order to acquire echo signals by the number of times of phase encoding. In a case where a reception coil includes a plurality of channels, the echo signals are acquired for each channel.

The k-space database 202 stores echo signal data of TE1 and TE2 respectively. By performing a Fourier transform on k-space data, two types of image data with different TEs are collected.

Although not illustrated in a drawing, the main measurement may use a frequency encoding gradient magnetic field of a polarity opposite to the frequency encoding gradient magnetic field used in FIG. 3. Specifically, after the frequency encoding gradient magnetic field (pre-pulse) 304 is applied in the positive direction, the frequency encoding gradient magnetic field 305 is applied in the negative direction, and then the frequency encoding gradient magnetic field 306 may be applied in the positive direction.

In case of the DIXON technique, TE1 can be set as a timing at which phases of echo signals (water signals) from water protons and echo signals (fat signals) from fat protons become reverse, and TE2 can be set as a timing at which phases of the water signals and the fat signals become the same. On the contrary to the above, it may be configured so that the water signals and the fat signals become the same phase in TE1 and the signals become the reverse phase in TE2. In order to reduce an imaging time, it is desirable to set the timings at which the water signals and the fat signals become the reverse phase in TE1 and the signals become the same phase in TE2.

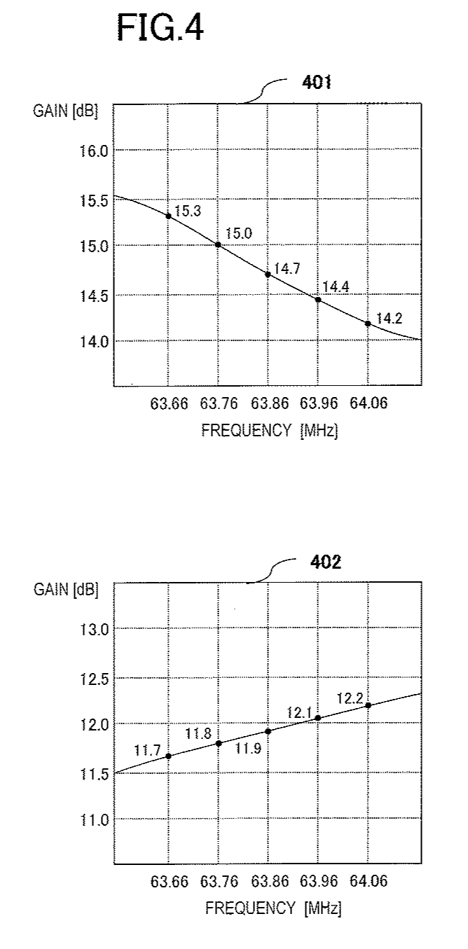

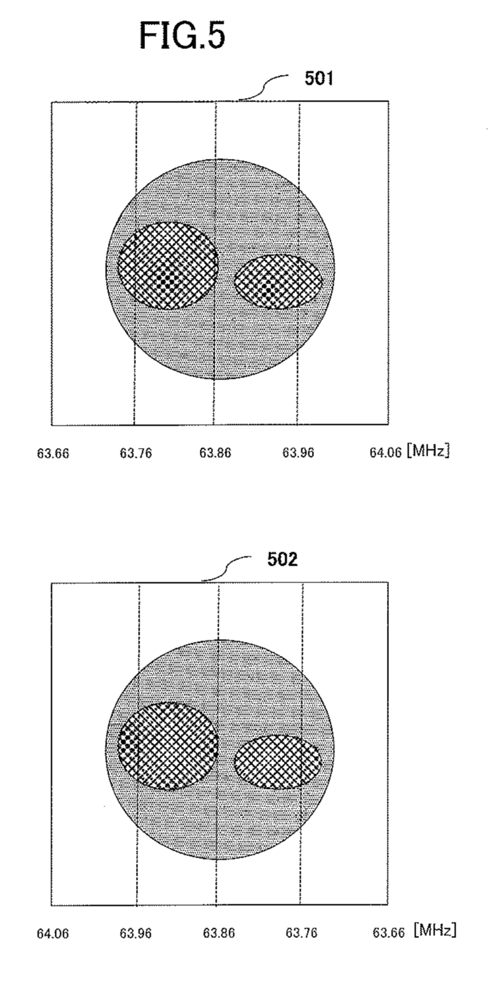

The adverse effect of reception frequency characteristics is found in the image data acquired thus. Here, the found an adverse effect of reception frequency characteristics will be described using FIGS. 4 and 5. FIG. 4 shows an example of reception frequency characteristics of a reception coil and illustrates the reception frequency characteristics of each channel of a two-channel coil. FIG. 5 illustrates the effect of the reception frequency characteristics on an image.

401 of FIG. 4 illustrates reception frequency characteristics of the channel number 1, and 402 illustrates reception frequency characteristics of the channel number 2. The Channel number 1 indicates a gain of 15.3 [dB] at 63.66 [MHz], and the gain decreases as the reception frequency increases, which results in 14.2 dB at 64.06 [MHz]. On the other hand, the Channel number 2 indicates a gain of 11.7 [dB] at 63.66 [MHz], and the gain increases as the reception frequency increases, which results in a gain of 12.2 [dB] at 64.06 [MHz].

When imaging is performed by setting a reception center frequency in a frequency encoding gradient magnetic field to 63.86 [MHz] and a reception band width of both the ends of FOV to 400 [KHz] using the reception coil having the reception frequency characteristics of FIG. 4, a positive-polarity image and a negative-polarity image have a relationship as illustrated in FIG. 5.

In FIG. 5, an image 501 illustrates a positive-polarity image, and an image 502 is a negative-polarity image. Becausethe images 501 and 502 have frequency encoding gradient magnetic fields in the opposite directions, a frequency increases from the left to the right in frequency encoding of the image 501, and a frequency decreases from the left to the right in frequency encoding of the image 502. For example, a case of an image based on signals acquired the channel number 1 will be described. As described in FIG. 4, a gain decreases as a reception frequency increases in the reception frequency characteristics of the channel number 1. That is, in the image 501, the gain of the reception frequency characteristics decreases as the frequency goes from the left to the right.

On the other hand, in the image 502, the gain of the reception frequency characteristics increases as the frequency goes from the left to the right. Specifically, the channel number I indicates a gain of 15.0 [dB] at 63.76 [MHz] (the point b) in the image 501 (refer to FIG. 4), and the gain decreases as the frequency goes from the left to the right (in other words, as the reception frequency increases), which results in a gain of 14.4 [dB] at 63.96 [MHz] (the point d) (refer to FIG. 4). On the other hand, a gain of the channel number 1 increases as the frequency goes from the left to the right (in other words, as the reception frequency decreases) in the image 502. Although no differences between the signal values are shown in the images of FIG. 5, when an effect of the reception frequency characteristics of the channel number 1 is reflected, a left signal value of the image 501 is higher than that of the image 502, and a right signal value of the image 501 is lower than that of the image 502 inversely.

On the other hand, a case of an image based on signals acquired the channel number 2 will be described. As described in FIG. 4, a gain of the reception frequency characteristics of the channel number 2 increases as a frequency increases. When an effect of the reception frequency characteristics of the channel number 2 is reflected, a left signal value of the image 501 is lower than that of the image 502, and a right signal value of the image 501 is higher than that of the image 502 inversely.

As described above, the two channels have the different reception frequency characteristics respectively, the effect of the reception frequency characteristics are generated differently for each channel.

Thus, the effects of the reception frequency characteristics of each of these channels are combined in an image combined from the signals acquired by a plurality of the channels. As the result, the accuracy of an image of fat contents deteriorates due to these effects.

Next, described will be a correction data measuring pulse sequence for removing an adverse effect of reception frequency characteristics. The correction data measuring pulse sequence inverts a frequency encoding gradient magnetic field in the main measurement pulse sequence and applies only a low-frequency phase encoding gradient magnetic field.

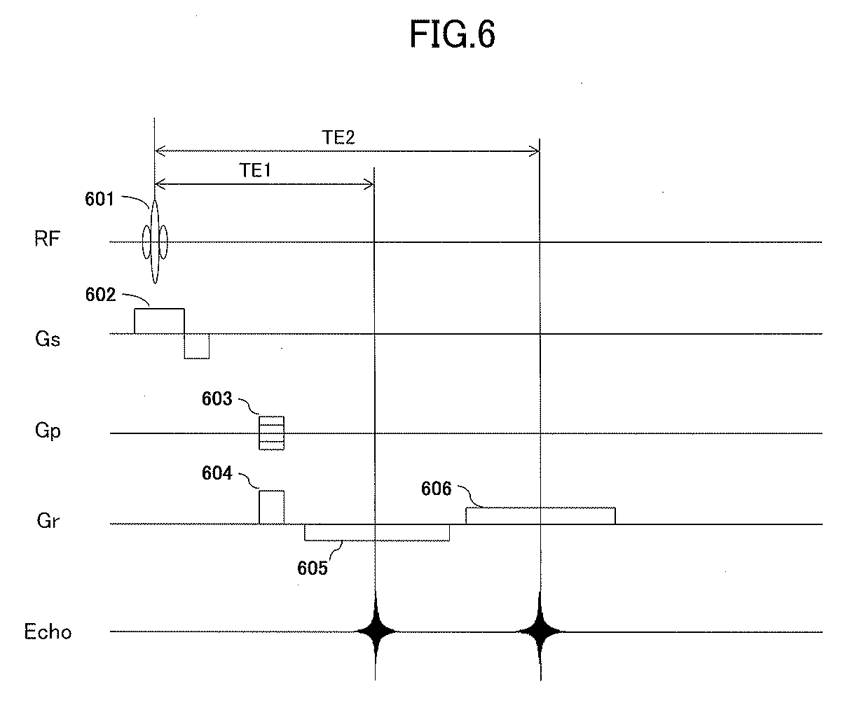

FIG. 6 shows an example of the correction data measuring pulse sequence. The correction data measuring pulse sequence is similar to the main measurement pulse sequence of FIG. 3 except that polarities of frequency encoding gradient magnetic fields 604, 605, and 606 are inverse of the polarities of the frequency encoding gradient magnetic fields 304, 305, and 306 in the main measurement pulse sequence respectively and that a phase encoding gradient magnetic field 603 is a phase encoding gradient magnetic field at low frequencies only. Such a sequence is repeatedly executed by the number of times of phase encoding at low frequencies only while changing an application amount of the phase encoding gradient magnetic field 603. Either of TE1 signals or TE2 signals acquired in this correction data measurement is used for correction.

In a case of the DIXON technique, in order to avoid canceling out water and fat signals, echo signals to be used for the correction are desirably acquired in a TE where water and fat phases are the closest to the same phase.

For example, in a case where TE2 is a timing when the water and fat signals are in the same phase in the main measurement pulse sequence of FIG. 3, used as correction data are an echo signal acquired from the same low-frequency phase encoding as the correction data measuring pulse sequence of FIG. 6 from among negative-polarity echo signals in TE2 of FIG. 3 in the main measurement and a positive-polarity echo signal in TE2 of FIG. 6 in the correction data measurement. Alternatively, in a case where TE1 is a timing when the water and fat signals are in the same phase in the main measurement pulse sequence of FIG. 3, used as correction data are an echo signal acquired in the same low-frequency phase encoding as the correction data measuring pulse sequence of FIG. 6 from among positive-polarity echo signals in TE1 of FIG. 3 in the main measurement and a negative-polarity echo signal in TE1 of FIG. 6 in the correction data measurement.

As described above, either of the two signals in the correction data measurement should be used. Therefore, although the correction data measuring pulse sequence illustrated in FIG. 6 illustrates a sequence to acquire the two echo signals from excitation by one RF pulse irradiation, one echo signal may be acquired from one excitation in the correction data measuring sequence.

The correction data measurement is performed by applying a low-frequency phase encoding gradient magnetic field that includes a zero phase encoding gradient magnetic field. Concerning an application amount of the low-frequency phase encoding gradient magnetic field, 8 is desirable, 16 is more desirable, and 32 is even more desirable.

The correction data measurement may be executed in the same slice position, in the same FOV (Field of View), in the same frequency encoding direction, by the same number of sampling points of frequency encoding, and in the same reception band width as main measurement immediately before or after the main measurement. Also, the correction data measurement may be executed continuously with or separately from the main measurement.

The correction data measurement is performed for each coil. For example, in a case of reception using a coil having a plurality of small coils (channels), the correction data measurement desirably acquires signals with each of the small coils. Also, the correction data measurement is desirably performed for each target to be imaged.

Echo signals acquired in the correction data measurement are stored in the correction database 203. Also, echo signals acquired by applying the same phase encoding gradient magnetic field as the low-frequency phase encoding gradient magnetic field in the correction data measuring pulse sequence from among the signals acquired in the main measurement are stored in the correction database 203 separately from the signals stored in the k-space database.

Hereinafter, based on the processing flow of FIG. 7, a correction process in the image conversion unit 204 will be described. Here, the description is made by taking a case of acquiring an Out-of-phase image in which water signals and fat signals are in the reverse phase by applying a positive-polarity frequency encoding gradient magnetic field and acquiring an In-phase image in which the water signals and the fat signals are in the same phase by applying a negative-polarity frequency encoding gradient magnetic field using the two-point DIXON technique as an example.

(Step S701)

A two-dimensional Fourier transform is performed on k-space data store in the k-space database 202 to convert the k-space data into images. The converted images are an Out-of-phase image acquired by applying a positive-polarity frequency encoding gradient magnetic field and an In-phase image acquired by applying a negative-polarity frequency encoding gradient magnetic field.

(Step S702)

The two-dimensional Fourier transform is performed respectively on echo data that is stored in the correction database 203 and in which polarities of frequency encoding gradient magnetic fields in order to convert the echo data into an image space. The correction data is echo signals acquired in a phase encoding gradient magnetic field at low frequencies only for one slice.

(Step S703)

A ratio is calculated from positive-polarity correction data and negative-polarity correction data on which the two-dimensional Fourier transform was performed respectively. One signals should be adjusted to the other signals so as to remove an adverse effect of reception frequency characteristics included in positive- and negative-polarity signals. As an example, described will be a calculation method of a correction data ratio for correcting so as to adjust the In-phase image acquired in application of the negative-polarity frequency encoding gradient magnetic field to the adverse effect of reception frequency characteristics included in the positive-polarity echo signals. When x is a frequency encoding direction; y is a coordinate in a phase encoding direction; positive-polarity correction data on which a Fourier transform was performed is Cp(x, y); and negative-polarity correction data on which a Fourier transform was performed is Cm(x, y), a ratio Cr(x) is calculated as follows.

$$\begin{matrix}[ Equation 1 ] & \\ Cr ( x ) = \frac{{\sum }_{y} \vert Cp ( x , y ) \vert}{{\sum }_{y} \vert Cm ( x , y ) \vert} & ( 1 )\end{matrix}$$

Here, “| |” shows an absolute value.

(Step S704)

Fitting is performed on a ratio of the correction data. Since the correction data includes noise, this is performed to remove a noise effect. Before fitting, the noise data is excluded by threshold processing. Because the adverse effect of reception frequency characteristics on an image induces such a variation like a linear or quadratic function on a signal, fitting of the correction data ratio should be also performed using the linear or quadratic function. The correction data ratio after fitting is shown with Fitting {Cr(x)}.

(Step S705)

Using the correction data Fitting {Cr(x)} on which fitting was performed, an image on which a two-dimensional Fourier transform was performed is corrected. In a case of correcting an In-phase image acquired by applying a negative-polarity frequency encoding gradient magnetic field and when a coordinate in a phase encoding direction is y; and the In-phase image is In(x, y), the corrected In-phase image In′(x, y) is calculated as follows.

[Equation 2]

In′(x, y)=In(x,y)×Fitting{Cr(x)} (2)

The corrected In-phase image In′(x, y) is consequently adjusted to the adverse effect of reception frequency characteristics included in echo signals acquired during application of a positive-polarity frequency encoding gradient magnetic field.

Steps S5701 to S705 are performed for each reception coil and each slice. Also, in a case where the coil is a reception coil having a plurality of small coils (channels), Steps S701 to S705 are performed for each small coil (channel) and each slice.

It is noted that the correction data ratio evaluated in Step S703 is an inverse of the right side of Equation (1) in a case of correcting a positive-polarity image. In Step S704, the image conversion unit 204 performs fitting on the inverse. In Step S705, the image conversion unit 204 multiplies the inverse on which fitting was performed by the image. The corrected image is consequently adjusted to the adverse effect of reception frequency characteristics included in negative-polarity signals.

Next, using a publicly known method, the image processing unit 206 uses the image corrected by the image conversion unit 204, combines images of each channel, and generates water images and fat images, which can perform a process to evaluate fat content ratios. FIG. 8 shows an example of such a process.

(Step S801)

Cases of combining In′(x, y) that is the In-phase image corrected in the image conversion unit 204 and combining Out(x, y) that is an out-of-phase image are illustrated. The combination of images of each channel is performed using the following equation.

$$\begin{matrix}[ Equation 3 ] & \\ {In}_{Comb} ( x , y ) = \frac{{\sum }_{k = 1}^{N} {In}_{k}^{′} ( x , y ) \times {M}_{k}^{*} ( x , y )}{\sqrt{{\sum }_{k = 1}^{N} {\vert {M}_{k} ( x , y ) \vert}^{2}}} & ( 3 ) \\ [ Equation 4 ] & \\ {Out}_{Comb} ( x , y ) = \frac{{\sum }_{k = 1}^{N} {Out}_{k} ( x , y ) \times {M}_{k}^{*} ( x , y )}{\sqrt{{\sum }_{k = 1}^{N} {\vert {M}_{k} ( x , y ) \vert}^{2}}} & ( 4 )\end{matrix}$$

|n_{comb}(x, y) is an image in which the corrected In-phase image In′(x, y) was combined. k indicates a channel number of the reception coil, and N indicates a channel number. Also, Mₖ(x, y) is a sensitivity map for combining the channels, and this is generated by providing a low-pass filter for In′(x, y). * indicates a complex conjugate.

Similarly, Out_{Comb}(x, y) is an image in which an Out-of-phase image Out(x, y) was combined. The sensitivity map to be used for the combination is the same as that for combining In_{Comb}(x, y).

(Step S802)

Generated is a phase map that illustrates phase variations to be generated due to static magnetic field inhomogeneity during different TEs. For example, in FIG. 3, TE1 is an Out-of-phase image, and TE2 is an In-phase image. First, a phase of the combined Out-of-phase image Out_{Comb}(x, y) is subtracted from the combined In-phase image In_{Comb}(x, y), and the phase is doubled to generate an initial phase map φ(x, y). Although water and fat are in the reverse phase in the Out-of-phase image, the reverse phase of water and fat is cancelled out by doubling the phase. This is expressed using Equation (5).

[Equation 5]

Φ(x, y)=Arg{(In_{Comb}(x, y)×Out*_{Comb}(x, y)) ²} (5)

Arg shows that an angle is evaluated from complex data. Next, a phase unwrapping process is performed on the initial phase map φ(x, y). The phase unwrapping process cancels out a point where phases are spatially discontinuous because ranges indicated by the phases are between −π to +π for spatial continuity. Because the phases are doubled in the initial phase map, the phase value is halved after phase unwrapping, and then the phase map φ(x, y) is completed. This is expressed using Equation (6).

$$\begin{matrix}[ Equation 6 ] & \\ φ ( x , y ) = \frac{1}{2} \times Wrap ( Φ ( x , y ) ) & ( 6 )\end{matrix}$$

Wrap shows phase unwrapping.

(Step S803)

A water image Water(x, y) and a fat image Fat(x, y) are generated using the combined In-phase image In_{Comb}(x, y), the combined Out-of-phase image Out_{Comb}(x, y), and the phase map. This is expressed using Equation (7).

$$\begin{matrix}[ Equation 7 ] & \\ Water ( x , y ) = \frac{{Out}_{Comb} ( x , y ) + {In}_{Comb} ( x , y ) \times exp ( - iφ ( x , y ) )}{2} \\ Fat ( x , y ) = \frac{- {Out}_{Comb} ( x , y ) + {In}_{Comb} ( x , y ) \times exp ( - iφ ( x , y ) )}{2} & ( 7 )\end{matrix}$$

(Step S804)

Fat content ratio images FatRatio(x, y) are generated using the water image Water(x, y) and the fat image Fat(x, y) or the In-phase image In_{Comb}(x, y) and the fat images Fat(x, y). This is expressed using Equation (8).

$$\begin{matrix}[ Equation 8 ] & \\ FatRatio ( x , y ) = \frac{abs \{ Fat ( x , y ) \}}{abs \{ {In}_{Comb} ( x , y ) \}} \\ FatRatio ( x , y ) = \frac{abs \{ Fat ( x , y ) \}}{abs \{ Water ( x , y ) \} + abs \{ Fat ( x , y ) \}} & ( 8 )\end{matrix}$$

In Equation 8, abs shows an absolute value.

This is also similar in a case where In-phase images In(x, y) and corrected Out-of-phase images Out′(x, y) are combined in order to respectively generate a water-fat separation image and a fat content ratio image.

The image transmission unit 207 transmits processed images to the display unit 111. The images include In-phase images, Out-of-phase images, water images, fat images, fat content ratio image, and the like.

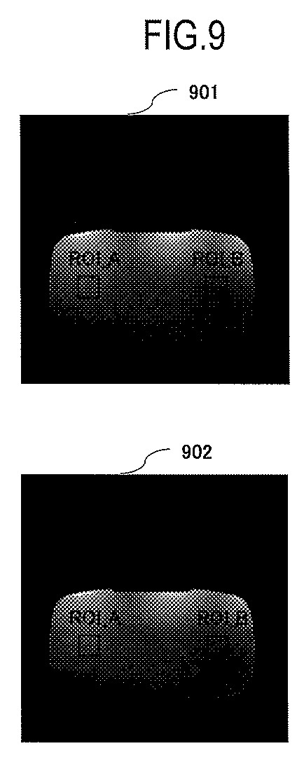

Hereinafter, an example of imaging with a phantom of an aqueous nickel chloride solution is used for describing that an adverse effect of reception frequency characteristics can be actually corrected by applying the above-described method of the first embodiment using signal values of echo images. FIG. 3 is used as a main measurement pulse sequence, and the pulse sequence illustrated in FIG. 6 is used as a correction data measuring sequence. Hereinafter, the results will be described using FIGS. 9 to 12.

Described first will be an image acquired by measurement in the main measurement pulse sequence and the adverse effect of reception frequency characteristics included in the image.

FIG. 9 illustrates images in which a phantom of an aqueous nickel chloride solution was imaged in the main measurement pulse sequence using four-channel reception coils under the conditions of FOV: 350 mm; reception band width: 360 kHz; the number of sampling in a frequency encoding direction: 256; and the number of sampling in a phase encoding direction: 128. An image 901 is an image (TE1 echo image) acquired in a state where the frequency encoding gradient magnetic field has a positive polarity (the frequency increase from the left to the right) and TE1 is 3.6 ms. On the other hand, an image 902 is an image (TE2 echo image) acquired in a state where the frequency encoding gradient magnetic field has a negative polarity (the frequency decrease from the left to the right) and TE2 is 4.9 ms.

The table 1 shows signal average values of ROI_A and ROI_B in the TE1 echo image 901, signal average values of ROI_A and ROI_B in the TE2 echo image 902, and signal average values of ROI_A and ROI_B in the TE2 echo image 902 in which an adverse effect of reception frequency characteristics was corrected. It is noted that coordinates of ROI_A and ROI_13 in each echo image are the same. A ROI_A value in the TE2 echo image 902 is less compared with the TE1 echo image 901. On the other hand, a ROI_B value in the TE2 echo image 902 is greater compared with the TE1 echo image 901. However, since it is obvious that there is T2* attenuation, a signal value of the TE2 echo image 902 in which the TE is 4.9 ms must be smaller than a signal value of the TE1 echo image 901 in which the TE is 3.6 ms. This is due to the adverse effect of reception frequency characteristics, and the TE2 echo image 902 needs to be corrected.

TABLE 1

| | Signal Average | Signal Average |
|---|---|---|
| | Value of ROI_A | Value of ROI_B |
| TE1 echo image | 89344 | 51648 |
| TE2 echo image | 87511 | 52010 |
| TE2 echo image in | 87191 | 49721 |
| which an adverse | | |
| effect of reception | | |
| frequency | | |
| characteristics was | | |
| corrected | | |

Hereinafter, described will be an example for correcting the TE2 echo image 902 so as to be adjusted to the adverse effect of reception frequency characteristics included in positive-polarity echo signals.

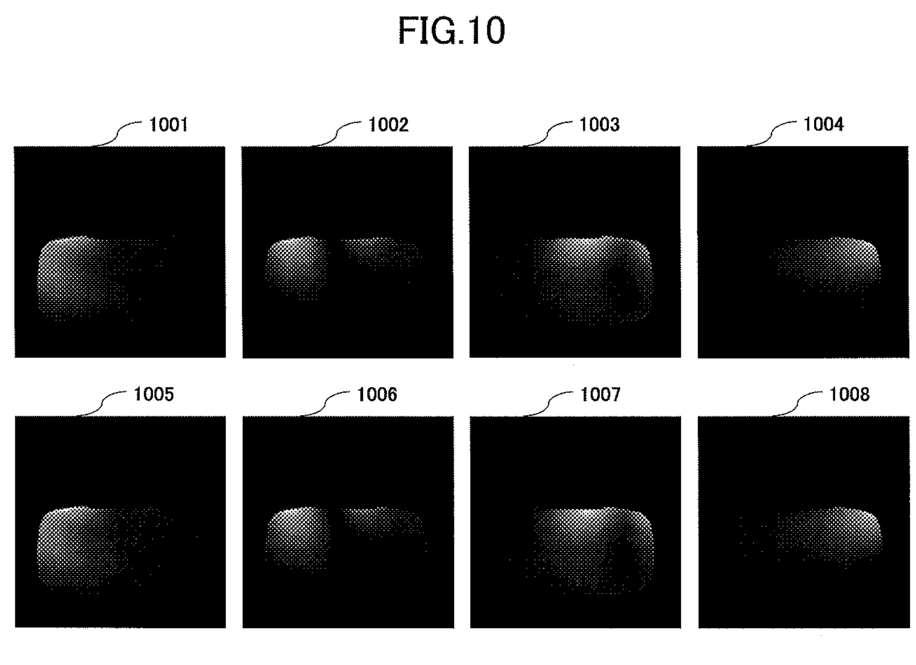

FIG. 10 illustrates images of each channel of the reception coils for the TE1 echo image 901 and the TE2 echo image 902 imaged in the main measurement. Images 1001 to 1004 are images of each channel of the reception coils for the TE1 echo image 901, and the images 1001, 1002, 1003, and 1004 are images of the channels 1, 2, 3, and 4 respectively. Also, images 1005 to 1008 are images of each channel of the reception coils for the TE2 echo image 902, and the images 1305, 1306, 1307, and 1308 are images of the channels 1, 2, 3, and 4 respectively.

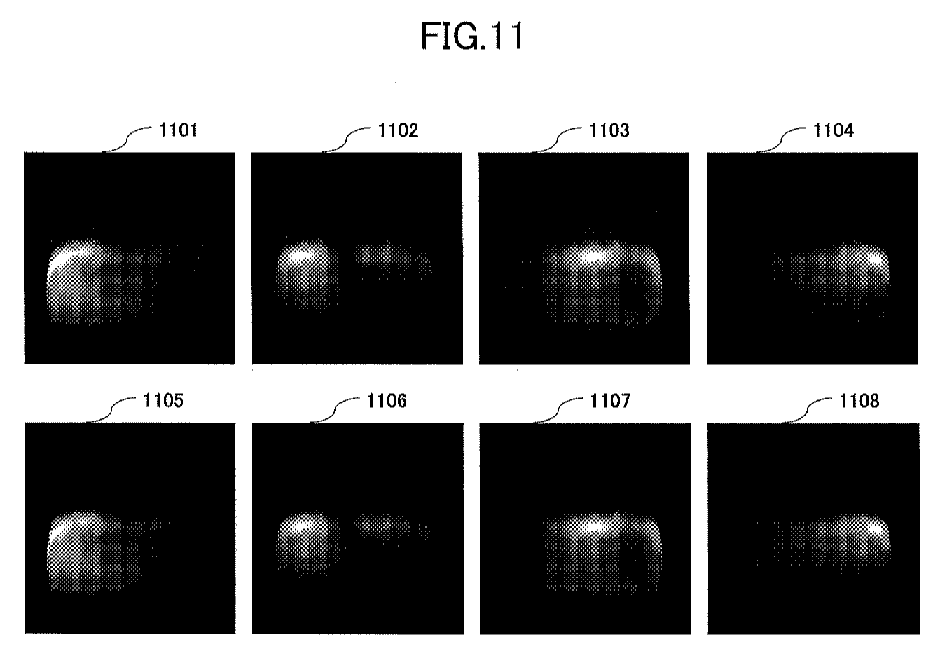

FIG. 11 shows correction data on which a Fourier transform was performed. Data (images 1101 to 1104) acquired by measuring a main measurement pulse sequence and data (images 1105 to 1108) acquired by measuring a correction data measuring pulse sequence were used as the correction data. Used were Echo signals of TE=TE1.

The correction data images 1101 to 1104 are correction data constructed from 16 phase encodes at a low frequency region of TE1 echo images acquired in the main measurement whose frequency encoding gradient magnetic field has a positive polarity.

The images 1101, 1102, 1103, and 1104 illustrate the channels 1, 2, 3, and 4 respectively.

Also, the images 1105, 1106, 1107, and 1108 are TE1 echo images acquired during application of a positive-polarity frequency encoding gradient magnetic field using the correction data measuring pulse sequence. The images 1105, 1106, 1107, and 1108 illustrate the channels 1, 2, 3, and 4 respectively. The phase encoding at a low frequency of 16 was used for a phase encoding gradient magnetic field in the correction data measuring pulse sequence.

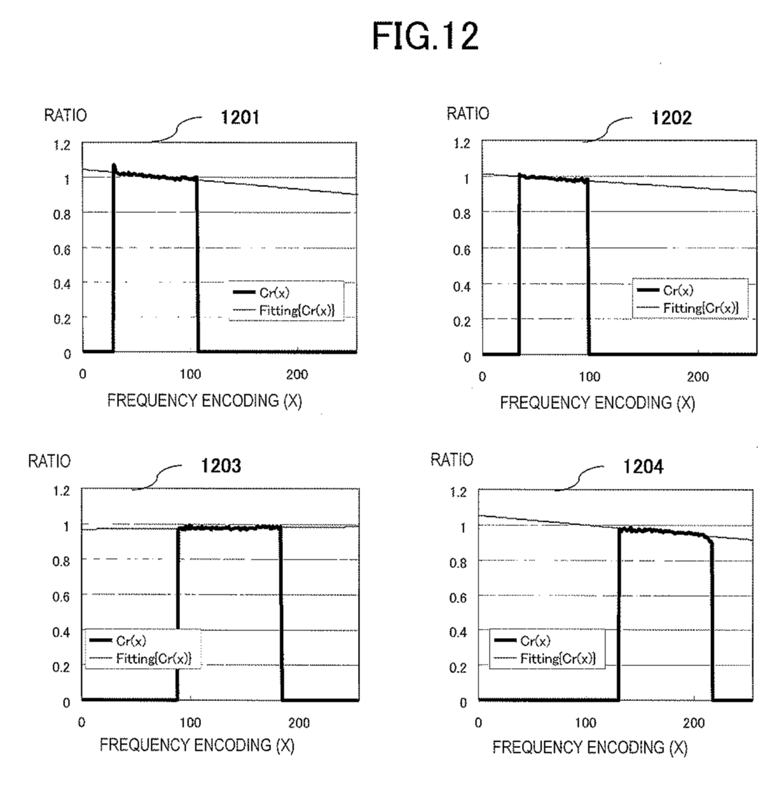

FIG. 12 shows CO) that is a correction data ratio and Fitting{Cr(x)} that is a correction data ratio on which fitting was performed using a linear function from the correction data shown in FIG. 11. Graphs 1201, 1202, 1203, and 1204 illustrate the channels 1, 2, 3, and 4 respectively. It is noted that the correction data ratios Cr(x) in the graphs 1201 to 1204 indicates only data that was used for fitting by threshold processing.

The images 1005 to 1008 of TE2 echo channels are corrected respectively with ratios Fitting{Cr(x)} of the correction data 1201 to 1204 on which fitting was performed, a channel-combined image is generated, and then values for ROI_A and ROI_B of the TE2 echo image 902 in which an adverse effect of reception frequency characteristics was corrected are calculated.

The calculated values are shown in the signal average values of ROI_A and ROI_B in the TE2 echo images in which the adverse effect of reception frequency characteristics was corrected in Table 1. In the TE2 echo images in which the adverse effect of reception frequency characteristics was corrected, the signal values of ROI_A and ROI_B are reasonable and attenuated by 3% compared with the TE1 echo images. Therefore, the adverse effect of reception frequency characteristics can be removed by the present embodiment.

A water image, a fat image, and a fat content ratio image evaluated from Out-of-phase images and In-phase images in which the reception frequency characteristics were corrected are acquired as above and have high accuracy because the effect of the reception frequency characteristics when a polarity of a frequency encoding gradient magnetic field is inverted has been corrected and removed, which reduces the error.

Effects of reception frequency characteristics in positive-polarity and negative-polarity images acquired by inverting a frequency encoding gradient magnetic field can be removed by the present embodiment. By removing the effects of reception frequency characteristics, accuracy of quantitative assessment of fat can be improved even in a case of using images acquired by imaging the hepatic region or the like at a high speed during respiratory arrest. Also, in imaging to acquire images for the quantitative assessment of fat, the imaging time can be reduced, or the number of imaging slices can be increased because there is no need to perform measurement in a frequency encoding gradient magnetic field of the same polarity in order to avoid the adverse effect of reception frequency characteristics.

### Second Embodiment

Although a second embodiment is the same as the first embodiment in executing a main measurement pulse sequence and a correction data measuring pulse sequence under control by the control unit 112, the second embodiment is different in separately acquiring and using two types of echo signals having the same TE as a main measurement and frequency encoding gradient magnetic fields of different polarities without using signals acquired in the main measurement as correction data.

The main measurement pulse sequence is the same as the main measurement pulse sequence (FIG. 3) described in the first embodiment.

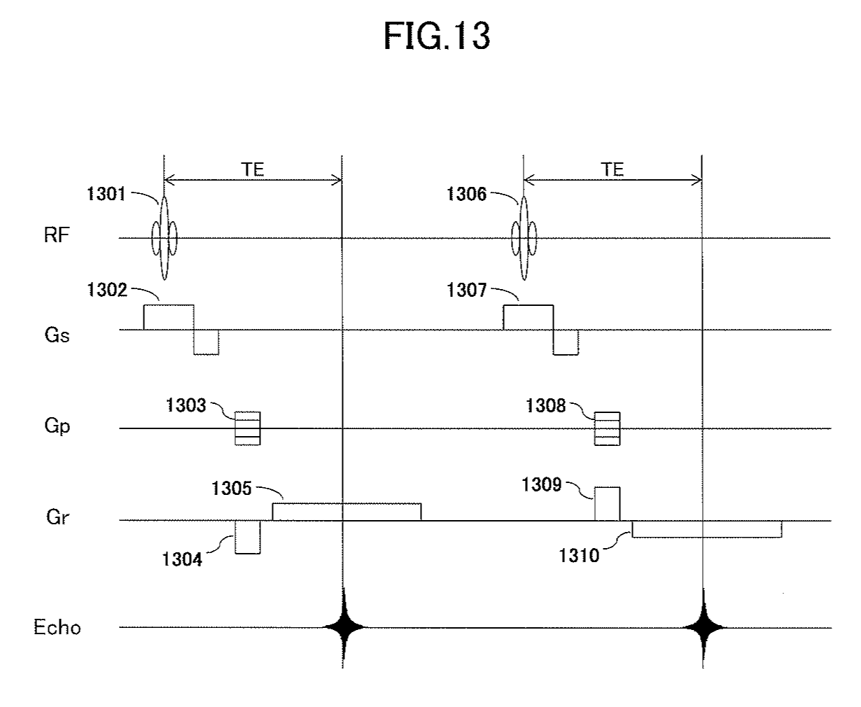

A correction data measuring sequence to be used for the present embodiment acquires one echo signal twice from excitation by irradiating one RF pulse. The two types of echo signals to be acquired are acquired during application of frequency encoding gradient magnetic fields of different polarities at the same echo time. FIG. 13 shows an example of the correction data measuring sequence to be used for the present embodiment. The correction data measuring sequence is a gradient echo (GrE)-type sequence. The type of the correction data measuring sequence is set to the same as that of a main measurement pulse sequence.

Only a desired tomographic plane is excited by applying a slice selection gradient magnetic field 1302 at the same time as first irradiation of an RF pulse 1301. Then, after simultaneously applying a phase encoding gradient magnetic field 1303 at low frequencies only for encoding positional information and a frequency encoding gradient magnetic field 1304 in the negative direction, a frequency encoding gradient magnetic field 1305 in the positive direction is applied in order to acquire echo signals to be generated after a TE elapses from the RF pulse as positive-polarity echo signals. Conditions after the next irradiation of an RF pulse 906 are set similarly to the above conditions for acquiring the positive-polarity echo signals other than applying a frequency encoding gradient magnetic field 1310 in the negative direction after applying a frequency encoding gradient magnetic field 1309 in the positive direction in order to acquire negative-polarity echo signals after the TE elapses. These positive- and negative-polarity echo signals are acquired at the same echo time.

In a case of the correction data measuring sequence of FIG. 9, although a TE having the same time may be set to either of two TEs in a main measurement pulse sequence, i.e. TE1 or TE2 of FIG. 3, TE=TE1 is desirable from the viewpoint of reducing time for correction data measurement. On the other hand, in a case where the main measurement pulse sequence is the two-point DIXON technique, it is desirable that a TE of the correction data measuring pulse sequence is a TE when water and fat signals are in the same phase in order to avoid cancelling out the water and fat signals. For example, TE=TE1 is set in a case where TE1 is a timing when the water and fat signals are in the same phase in the main measurement. TE=TE2 is set in a case where TE2 is a timing when the water and fat signals are in the same phase in the main measurement.

Correction data measurement is performed by applying a low-frequency phase encoding gradient magnetic field including a zero phase encoding gradient magnetic field. Concerning an application amount of the low-frequency phase encoding gradient magnetic field, 8 is desirable, 16 is more desirable, and 32 is even more desirable.

The correction data measurement may be executed in the same slice position, in the same FOV (Field of View), in the same frequency encoding direction, by the same number of sampling points of frequency encoding, and in the same reception band width as main measurement immediately before or after the main measurement. Also, the correction data measurement may be executed continuously with or separately from the main measurement.

The correction data measurement is performed for each coil. For example, in a case of receiving using a coil having a plurality of small coils (channels), it is desirable to acquire signals for each of the small coils (channels). Also, it is desirable that the correction data measurement is performed for each target to be imaged.

A pair of positive- and negative-polarity echo signals acquired in the correction data measurement is stored in the correction database 203.

The process in which the image conversion unit 204 removes an adverse effect of reception frequency characteristics using correction data, the process in which the image processing unit 206 combines images of each channel and generates water and fat images in order to evaluate a fat content ratio using corrected images, and the process in which the image transmission unit 207 transmits image-processed images to the display unit 111 are similar to the first embodiment.

The present embodiment obtains the effect similar to the first embodiment by generating correction data using a pair of correcting echo signals acquired during application of frequency encoding gradient magnetic fields of different polarities at the same echo time by correction data measurement in order to correct echo signals acquired in main measurement.

### Third Embodiment

Although the sequence for acquiring two images with different TEs was used as main measurement in the first and second embodiments, a third embodiment will describe that a sequence for acquiring three images with different TEs can be applied.

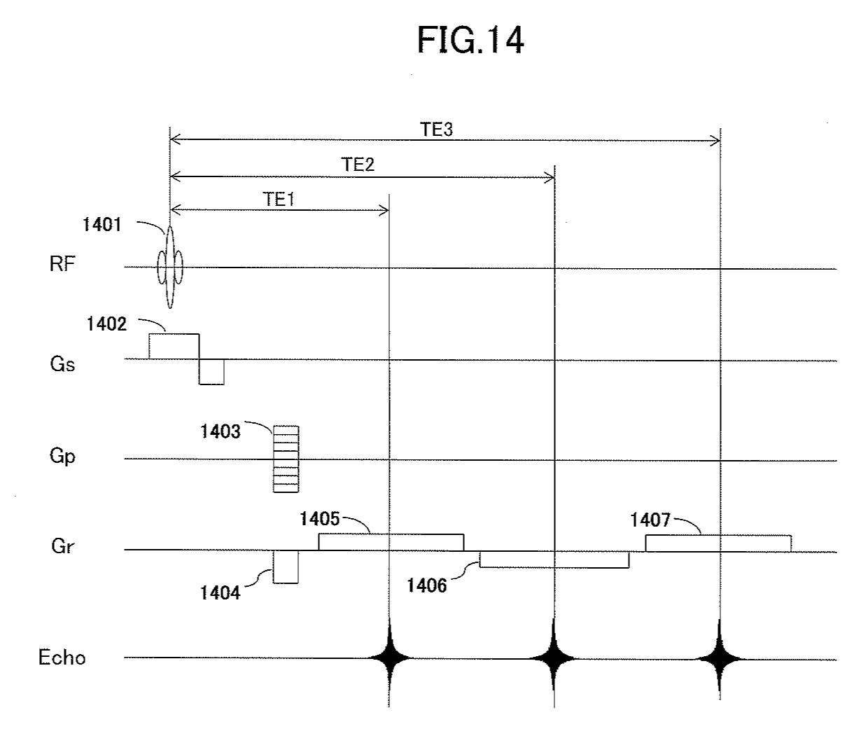

FIG. 14 shows an example of the sequence for acquiring three images with different TEs (a main measurement pulse sequence of the present embodiment). For example, this sequence can be used for a three-point DIXON technique.

The main measurement pulse sequence of FIG. 14 is similar to the main measurement pulse sequence shown in FIG. 13 other than applying a positive-polarity frequency encoding gradient magnetic field 1407 again in order to further acquire third echo signals and generating the third echo signals after TE3 elapses from the RF pulse. Three types of image data with different TEs can be acquired from the echo signals acquired by the main measurement pulse sequence of FIG. 10.

Also in a case of correcting image data acquired in the main measurement, the correction data may be acquired by only one set of positive-polarity and negative-polarity echo signals in the same TE as described in the first and second embodiments. As described in the first and second embodiments, the process of removing an adverse effect of reception frequency characteristics using the correction data by the image conversion unit 204, the process of combining images of each channel and generating water and fat images in order to evaluate a fat content ratio using the images corrected by the image processing unit 206, and the process of transmitting the image-processed images to the display unit 111 by the image transmission unit 207 may be performed.

According to the present embodiment, the same effect as the first and second embodiments can be acquired also when the main measurement uses the sequence for acquiring three images with different TEs.

Although the respective embodiments of the present invention are described above by taking the DIXON techniques as an example, the present invention can be applied to a three-dimensional gradient echo technique, a spin echo technique, a fast spin echo technique, and the like using a pulse sequence for acquiring a plurality of signals with different TEs. The present invention can be applied also in a case of using images acquired by performing high-speed imaging for regions of the other internal organs to which fat sticks other than the liver such as fat around the heart or visceral fat.

Although some embodiments of the present invention are described above, the present invention can remove effects of reception frequency characteristics in positive-polarity and negative-polarity images acquired by inverting a frequency encoding gradient magnetic field. By removing the adverse effect of reception frequency characteristics, provided can be an MRI apparatus that can improve accuracy of quantitative assessment of fat even in a case of using images acquired by imaging the hepatic region or the like at a high speed during respiratory arrest. Also, in imaging to acquire images for the quantitative assessment of fat, the imaging time can be reduced, or the number of imaging slices can be increased using the present invention because there is no need to acquire signals by applying a frequency encoding gradient magnetic field of the same polarity in order to avoid the adverse effect of reception frequency characteristics.

### INDUSTRIAL APPLICABILITY

The present invention can remove effects of reception frequency characteristics in positive-polarity and negative-polarity images by inverting a frequency encoding gradient magnetic field. By removing the effects of reception frequency characteristics, for example, accuracy of quantitative assessment of fat can be improved even in a case of using images acquired by imaging the hepatic region or the like at a high speed during respiratory arrest. Also, the imaging time can be reduced, or the number of imaging slices can be increased in imaging to acquire images for the quantitative assessment of fat.

### REFERENCE SIGNS LIST

101: object

102: static magnetic field magnet

103: gradient magnetic field coil

104: irradiation coil (high-frequency generating unit)

105: reception coil (reception unit)

106: bed

107: gradient magnetic field power source

108: RF transmission unit (high-frequency generating unit)

109: signal detection unit (reception unit)

110: signal processing unit

111: display unit

112: control unit

113: input unit

201: signal reception unit

202: k-space database

203: correction database

204: image conversion unit

205: image database

206: image processing unit

207: image transmission unit

208: memory (parameters)

301: RF pulse

302: slice selecting gradient magnetic field

303: phase encoding gradient magnetic field

304: frequency encoding gradient magnetic field (pre-pulse)

305: frequency encoding gradient magnetic field

306: frequency encoding gradient magnetic field

401: reception frequency characteristics of the channel number 1

02: reception frequency characteristics of the channel number 2

501: positive-polarity image

502: negative-polarity image

601: RF pulse

602: slice selecting gradient magnetic field

603: phase encoding gradient magnetic field

604: frequency encoding gradient magnetic field (pre-pulse)

605: frequency encoding gradient magnetic field

606: frequency encoding gradient magnetic field

901: TE1 echo image (positive-polarity image)

902: TE2 echo image (negative-polarity image)

1001 to 1004: TE1 echo images of each channel

1005 to 1008: TE2 echo images of each channel

1101 to 1104: correction data images

1105 to 1108: correction data images

1201 to 1204: graphs of correction data ratios of each channel

1301: RF pulse

1302: slice selection gradient magnetic field

1303: phase encoding gradient magnetic field

1304: frequency encoding gradient magnetic field (pre-pulse)

1305: frequency encoding gradient magnetic field

1306: RF pulse

1307: slice selection gradient magnetic field

1308: phase encoding gradient magnetic field

1309: frequency encoding gradient magnetic field (pre-pulse)

1310: frequency encoding gradient magnetic field

1401: RF pulse

1402: slice selection gradient magnetic field

1403: phase encoding gradient magnetic field

1404: frequency encoding gradient magnetic field (pre-pulse)

1405: frequency encoding gradient magnetic field

1406: frequency encoding gradient magnetic field

1407: frequency encoding gradient magnetic field

1. A magnetic resonance imaging apparatus comprising:
static magnetic field magnets; high-frequency generating units that generate high-frequency magnetic field pulses; reception units that include high-frequency coils receiving echo signals to be generated by nuclear magnetic resonance; gradient magnetic field coils; a control unit that controls the high frequency generation units, the gradient magnetic field coils, and the reception units according to a predetermined pulse sequence; and a signal processing unit that processes the echo signals,
wherein the pulse sequence is a multi-echo sequence that acquires echo signals during application of frequency encoding gradient magnetic fields of different polarities at a plurality of echo times after being excited by the high-frequency magnetic field pulses, and
wherein the signal processing unit generates correction data using a pair of correcting echo signals acquired during the application of frequency encoding gradient magnetic fields of positive and negative polarities at the same echo time and is provided with a correction unit that corrects echo signals acquired during the application of frequency encoding gradient magnetic fields of different polarities.

2. The magnetic resonance imaging apparatus according to claim 1,
wherein the correction unit generates the correction data using echo signals acquired in a correction data measuring sequence to be executed separately from the multi-echo sequence.

3. The magnetic resonance imaging apparatus according to claim 2,
wherein the correction data measuring sequence is the same type as the multi-echo sequence other than an application condition of a phase encoding gradient magnetic field.

4. The magnetic resonance imaging apparatus according to claim 1,
wherein the pair of correcting echo signals is echo signals acquired in the executed multi-echo sequence and echo signals acquired in a correction data measuring sequence to be executed separately from the multi-echo sequence.

5. The magnetic resonance imaging apparatus according to claim 4,
wherein the pair of correcting echo signals is signals acquired by applying a low-frequency phase encoding gradient magnetic field.

6. The magnetic resonance imaging apparatus according to claim 1,
wherein the high-frequency coil comprises a plurality of small coils, and
wherein the correction unit generates correction data for each of the small coils in order to perform correction on echo signals received by each of the small coils using the correction data.

7. The magnetic resonance imaging apparatus according to claim 6,
wherein the correction unit evaluates the correction data from a ratio of data acquired by performing a two-dimensional Fourier transform on the pair of correcting echo signals for each of the small coils.

8. The magnetic resonance imaging apparatus according to claim 1,
wherein the multi-echo sequence is a water-fat separation sequence that acquires echo signals at a first echo time in which echo signals from water and echo signal from fat are in the same phase and at a second echo time in which the echo signals from water and the echo signals from fat are in the reverse phase in the frequency encoding gradient magnetic fields of different polarities,

9. The magnetic resonance imaging apparatus according to claim 8,
wherein the echo signals used for generating the correction data is echo signals acquired at an echo time same as the first echo time or the second echo time.

10. The magnetic resonance imaging apparatus according to claim 8,
whereat the first echo time is set longer than the second echo time in the water-fat separation sequence.

11. A water-fat separation image generating method for generating a plurality of types of images using echo signals to be generated by nuclear magnetic resonance,
wherein the echo signals are acquired during application of frequency encoding gradient magnetic fields of different polarities at a plurality of echo times after being excited by a high-frequency magnetic field pulse, and
wherein the echo signals acquired during the application of frequency encoding gradient magnetic fields of different polarities are corrected using a pair of correcting echo signals acquired during application of frequency encoding gradient magnetic fields of positive and negative polarities at the same echo time.

12. The water-fat separation image generating method according to claim 11,
wherein the pair of correcting echo signals is signals acquired by applying a low-frequency phase encoding gradient magnetic field.

13. The water-fat separation image generating method according to claim 11,
wherein the echo signals are collected at the first echo time in which echo signals from water and echo signals from fat are in the same phase and at the second echo time in which the echo signals from water and the echo signals from fat are in the reverse phase, and
wherein a plurality of types of images are generated using first echo signals acquired at the first echo time and second echo signals acquired at the second echo time.

14. The water-fat separation image generating method according to claim 13,
wherein the pair of correcting echo signals is signals acquired by applying a low-frequency phase encoding gradient magnetic field at an echo time same as the first echo time or the second echo time.

15. The water-fat separation image generating method according to claim 11,
wherein a fat distribution ratio is calculated using the plurality of types of images.
